(19) United States
(12) Patent Application Publication
(10) Pub. No.: US 2013/0230475 A1
(43) Pub. Date: Sep. 5, 2013

(54) ANTIPERSPIRANT EMULSION PRODUCTS AND PROCESSES FOR MAKING THE SAME

(71) Applicant: Berea Thompson, Somerset, NJ (US)

(72) Inventor: Berea Thompson, Somerset, NJ (US)

(73) Assignee: The Dial Corporation, Scottsdale, AZ (US)

(21) Appl. No.: 13/409,677

(22) Filed: Mar. 1, 2012

(51) Int. Cl. A61K 8/34; A61Q 15/00

(52) U.S. Cl. 424/65

(57) ABSTRACT

A water in oil emulsion composition includes an antiperspirant active, an emulsifier, and a combination of polyalkylene glycols making up between about 1 and about 12 wt. % of the total composition. The combination includes H[OCH₂CH₂]ₘOH (I), and H[OCHCH₃CH₂]ₙOH (II) in a ratio I:II ranging between about 1.5:1 and about 1:1.5.

### FIELD OF THE INVENTION

The present invention generally relates to antiperspirant products and processes for making them, and more particularly relates to antiperspirant emulsion products that exhibit antiperspirant efficacy with the enhanced feel of a deodorant and processes for making them.

### BACKGROUND OF THE INVENTION

Antiperspirants and deodorants are popular personal care products used to prevent or eliminate sweat and body odor caused by sweat. Antiperspirants typically prevent the secretion of sweat by blocking or plugging sweat-secreting glands, such as those located at the underarms. Deodorants counteract or mask the unwanted odors caused by bacterial flora in secreted sweat.

In order to be effective underarm sweat inhibitors, antiperspirant salts are typically included in antiperspirant products at relatively high concentrations. These water-soluble salts, in concentrated solutions, are sticky as they dry, thereby producing an unpleasant tacky feeling in the underarms. A variety of product forms have been developed with the sole purpose of dealing with this problem. For example, the problem of tackiness is largely overcome with formulations incorporating a solid fatty stick that could be rubbed into the underarm area. These forms still have some significant degree of consumer acceptance, but they are being rejected by many consumers because both forms tend to deposit a white, powdery residue in the axilla. It is particularly difficult to overcome tackiness in formulations incorporating water-in-oil suspensions of antiperspirant salts.

Accordingly, it is desirable to provide antiperspirant formulations that incorporate water-in-oil suspensions that exhibit adequate antiperspirant efficacy but do not impart a sticky or tacky feeling to the user. In addition, it is desirable to provide processes for making such antiperspirant products. Furthermore, other desirable features and characteristics of the present invention will become apparent from the subsequent detailed description of the invention and the appended claims, taken in conjunction with the accompanying drawings and this background of the invention.

### BRIEF DESCRIPTION OF THE DRAWINGS

FIG. 1 depicts a film of water formed along the axilla of a user of an antiperspirant emulsion product, the water originating from the aqueous portion of the emulsion; and

FIG. 2 depicts a film of water formed along the axilla of a user of the antiperspirant emulsion product according the present invention, the water originating from the aqueous portion of the emulsion.

### BRIEF SUMMARY OF THE INVENTION

In accordance with an exemplary embodiment, a water in oil antiperspirant composition is provided. The antiperspirant product includes an antiperspirant active, an emulsifier, and a combination of polyalkylene glycols making up between about 1 and about 12 wt. % of the total composition, the combination comprising H[OCH₂CH₂]ₘOH (I), and H[OCHCH₃CH₂]ₙOH (II) in a ratio I:II ranging between about 1.5:1 and about 1:1.5.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

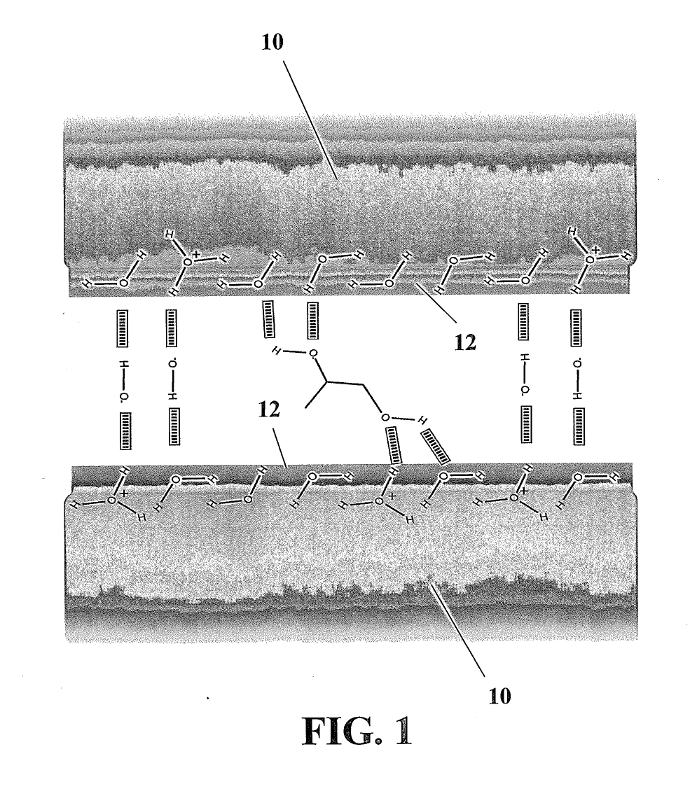

The various embodiments contemplated herein relate to antiperspirant emulsion products with antiperspirant efficacy that exhibit reduced tackiness caused by a high amount of hydrogen bonding between the hydroxyl groups from the acidic antiperspirant active and the skin surface. Without being bound to any theory or hypothesis, the present inventor conjectures that tackiness is perceived by the user due to hydrogen bonding between hydrophilic molecules in the emulsion product and water molecules and associated ions that form a film along the axilla upon application of the product. For example, hydrophilic carriers such as glycols are hydrogen bonding molecules. Referring to FIG. 1, a film of water 12 is formed along the axilla 10, the water originating from the aqueous portion of the antiperspirant emulsion product. Propylene glycol is a common carrier in such an emulsion, and a molecule of propylene glycol forms four hydrogen bond interactions with water and associated ions in the film of water 12. Likewise, metals such as aluminum and zirconium from antiperspirant actives are surrounded by water molecules and hydroxide ions in an aqueous emulsion. The force of separating the hydrogen bonds when the user moves his or her arms imparts a feeling of uncomfortable tackiness.

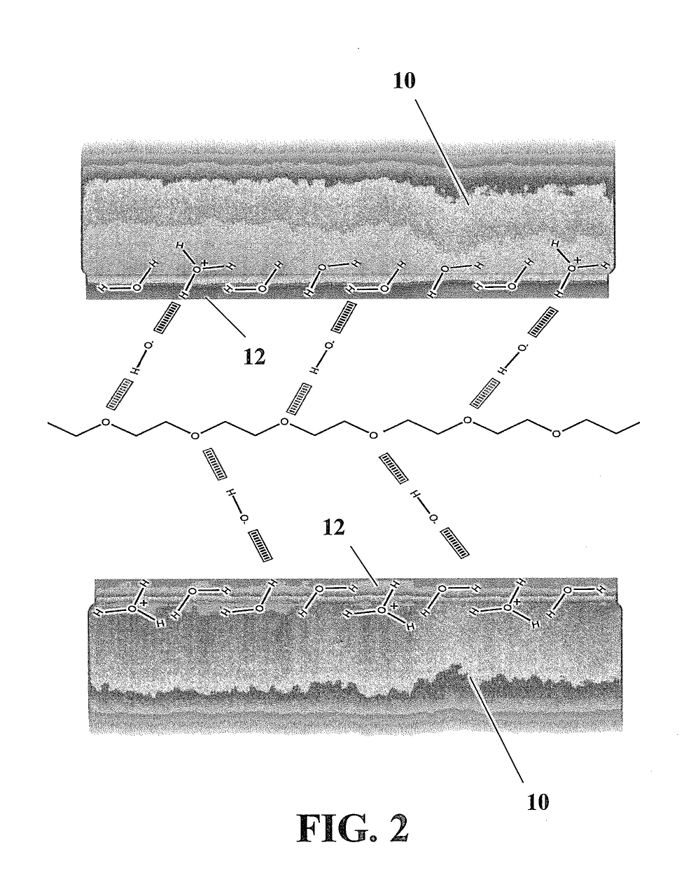

Exemplary antiperspirant emulsion products according to the present invention decrease or eliminate tackiness. This is achieved by providing antiperspirant emulsion formulations that include a combination of polyalkylene glycol components that have hydrogen bonding capacity coupled with an ability to orient water molecules in a manner that decreases the net hydrogen bonding energy with water molecules adhered to the axilla. Referring now to FIG. 2, the present inventor further conjectures that largely or entirely replacing polyethylene with longer chain polyalkylene glycols as carrier molecules in an antiperspirant emulsion formulation effectively reduces tackiness. This is because the repeating ether bonds in polyalkylene glycols, such as polyethylene glycol or polypropylene glycol, have more hydrogen bonding capability due to increased number of oxygen atoms per molecule, however, the ether hydrogen bond is somewhat weaker and relatively less force is necessary to dissociate the ether-hydroxide bonds.

The present inventor has also made the unexpected discovery that specific combinations of polyalkylene glycols impart a substantially reduced feeling of tackiness to a user. Furthermore, a surprising reduction in tackiness is achieved by a specific ratio between the different polyalkylene glycols, as will be explained hereinafter.

In this regard, in one exemplary embodiment of the present invention, the antiperspirant emulsion product, hereinafter referred to as the antiperspirant product, is a water-in-oil emulsion comprising a water phase mixed with an oil phase. Preferably, the antiperspirant product includes an oil phase in an amount of about 20 to about 45 weight percent (wt. %) of the total antiperspirant product and a water phase in an amount of about 55 to about 80 wt. % of the total antiperspirant product. The antiperspirant product preferably has a soft, non-flowing, solid composition that can be rubbed or wiped across the skin, particularly the underarm. However, the various embodiments are not so limited and the antiperspirant product can also have a gel, cream, or lotion consistency. The solid composition is substantially snow white in color, thus suggesting a clean and/or sterile nature.

The oil phase of the antiperspirant product includes at least one structurant and/or gellant (hereinafter referred collectively as structurant) that facilitates the solid consistency of the antiperspirant stick product. Naturally-occurring or synthetic waxy materials or combinations thereof can be used as such structurants. Examples of these waxy materials include those fatty alcohols that are solid at room temperature and hydrocarbon waxes or silicone waxes. Such materials are widely available, and by suitable selection of the materials themselves and their concentrations in the formulation, it is possible to obtain either a soft solid or a firm solid. In a preferred embodiment, the oil phase includes a high molecular weight (MW) polyethylene. As used herein, the term “high molecular weight (MW) polyethylene” means polyethylene having a molecular weight of 200 to 5000 daltons (Da). In a more preferred embodiment, the oil phase includes high MW polyethylene having a molecular weight of about 500 Da. In another preferred embodiment, the oil phase includes high MW polyethylene in an amount of about 5 to about 12 wt. % of the total antiperspirant product.

In accordance with another exemplary embodiment, when high MW polyethylene is used in the oil phase as a structurant, the oil phase also includes at least one low MW synthetic wax. In addition to facilitating the high MW polyethylene by serving a structurant function, the low MW synthetic wax also improves the manufacturing processes of the antiperspirant products. Generally, polyethylene has a relatively high melting point (70-100° C.) and, thus, as described in more detail below, the oil phase of the antiperspirant product must be heated to this high melting point to melt the polyethylene. However, this high temperature heating may result in higher manufacturing costs and unpredictable and/or non-repeatable yields of the antiperspirant product. The presence of an effective amount of low MW synthetic wax (synthetic wax having a molecular weight in the range of 1200-2900 Da) modifies the high MW polyethylene, lowering the melting point of the polyethylene. Accordingly, the presence of the low MW synthetic wax may result in lower manufacturing costs and higher yield of the antiperspirant product. In an exemplary embodiment, the low MW synthetic wax is present in the oil phase in an amount of about 0 to about 3 wt. % of the total antiperspirant product. In another exemplary embodiment, the low MW synthetic wax has a molecular weight of about 1800. In addition to improving hardness of the antiperspirant stick product, the low MW synthetic wax reduces syneresis and tackiness and also has a high refractive index (R.I.) that minimizes and/or prevents a white residue on the skin by masking the antiperspirant metallic salt(s) that stays upon the skin upon evaporation of the carrier(s), described in more detail below. As used herein, the term “high refractive index” means an refractive index no less than about 1.4. While the use of low MW synthetic wax to lower the melting point of high MW polyethylene is described herein.

The oil phase further includes at least one hydrophobic carrier. An example of suitable hydrophobic carriers includes liquid siloxanes and particularly volatile polyorganosiloxanes, that is, liquid materials having a measurable vapor pressure at ambient conditions. The polyorganosiloxanes can be linear or cyclic or mixtures thereof. Preferred siloxanes include cyclomethicones, such as cyclotetramethicone, cyclopentamethicone, and cyclohexamethicone, and mixtures thereof. The carrier also may include, additionally or alternatively, nonvolatile silicones such as dimethicone and dimethicone copolyols. Examples of suitable dimethicone and dimethicone copolyols include polyalkyl siloxanes, polyalkylaryl siloxanes, and polyether siloxane copolymers.

The oil phase may also include a high R.I. hydrophobic compound as a residue masking agent. The high R.I. hydrophobic compound minimizes and/or prevents a white residue on the skin by masking the antiperspirant metallic salt that stays upon the skin upon evaporation of the carrier(s). Examples of high R.I. hydrophobic compounds for use in the antiperspirant products include C₁₂-C₁₅ alkyl benzoate, such as Finsolv TN® available from Innospec of the United Kingdom, PPG-14 butyl ether, and phenyl dimethicone. In a preferred embodiment, the oil phase includes C₁₂-C₁₅ alkyl benzoate and, in a more preferred embodiment, the oil phase includes C₁₂-C₁₅ alkyl benzoate in an amount of about 2 to about 12 wt. % of the total antiperspirant product. High R.I. compounds such as these also provide beneficial emollients to improve skin feel properties and improve overall glide of the emulsion stick when applied to the user's skin.

The various embodiments of the antiperspirant emulsion products include at least one emulsifier. Emulsifying surfactants are employed in the antiperspirant product to facilitate the formation of a stable emulsion containing the water and oil phases. The emulsifying surfactants may be anionic, cationic, zwitterionic and/or nonionic surfactants. Nonionic surfactants are preferred in the present product. The proportion of emulsifier in the product is often selected in the range of up to 10% by weight and in many cases between 0.1 and 3% by weight. Emulsifiers are frequently classified by their hydrophilic-lipophilic balance (HLB) value. It is desirable, although not essential, to use an emulsifier or a mixture of emulsifiers with an overall HLB value that ranges between 2 and 10, preferably between 3 and 8.

It may be convenient to use a combination of two or more emulsifiers that have different HLB values above and below the desired value. By employing two emulsifers together in an appropriate ratio, it is readily feasible to attain a weighted average HLB value that promotes the formation of an emulsion.

Many suitable emulsifiers of high HLB are nonionic ester or ether emulsifers comprising a polyoxyalkylene moiety, especially a polyoxyethylene moiety, often containing from about 2 and 30, and preferably between 5 and 60 oxyethylene units, and/or contain a polyhydroxy compound such as glycerol or sorbitol or other alditol as a hydrophilic moiety. The hydrophilic moiety may contain polyoxypropylene. The emulsifiers additionally comprise a hydrophobic alkyl, alkenyl or aralkyl moiety, normally having from about 3 to about 50 carbons and particularly between about 10 and about 30 carbons. The hydrophobic moiety may be either linear or branched and is often saturated, though it may be unsaturated, and is optionally fluorinated. The hydrophobic moiety may comprise a mixture of chain lengths, for example, those deriving from tallow, lard, palm oil, sunflower seed oil, or soya bean oil. Such nonionic surfactants can also be derived from a polyhydroxy compound such as glycerol or sorbitol or other alditos. Examples of emulsifiers include ceteareth-10-25, ceteth-10-25, steareth-10-25 (i.e. C16-C18 alcohols ethoxylated with 10 to 25 ethylene oxide residues) and PEG-15-25 stearate or distearate. Other suitable examples include C10-C20 fatty acid mono, di or tri-glycerides. Further examples include C18-C22 fatty alcohol ethers of polyethylene oxides (8 to 12 EO).

Examples of emulsifiers that typically have a low HLB value, often a value from 2 to 6, are fatty acid mono or possibly diesters of polyhydric alcohols such as glycerol, sorbitol, erythritol or trimethylolpropane. The fatty acyl moiety is often from C14 to C22 and is saturated in many instances, including cetyl, stearyl, arachidyl and behenyl. Examples include monoglycerides of palmitic or stearic acid, sorbitol mono or diesters of myristic, palmitic or stearic acid, and trimethylolpropane monoesters of stearic acid.

A particularly desirable class of emulsifiers comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers also include C1 to C22 alkyl groups as functional groups. Examples of suitable surfactants include DC525 and DC5200 (from Dow Corning), Abil EM 90 and EM 97 (from Gold Schmidt) and KF 6026, KF 6028, and KF 6038 (from Shinetsu Silicones).

One preferred emulsifier for use in the antiperspirant formulations is cetyl PEG/PPG-10/1 dimethicone as it imparts a benefit to the antiperspirant product to exhibit skin feel characteristics that are typical of deodorant products. For example, with cetyl PEG/PPG-10/1 dimethicone, the antiperspirant emulsion products glide onto skin with less friction, that is, in a smoother manner, than conventional antiperspirants while still maintaining a solid consistency for easy application. In addition, when applied, the antiperspirant products are moister than typical antiperspirants and thus give the skin a moister and softer feel. In a preferred embodiment, the oil phase includes cetyl PEG/PPG-10/1 dimethicone in an amount of about 1 to about 4 wt. % of the total antiperspirant product.

The water phase of the antiperspirant product includes water, polyalkylene glycols, and a water-soluble active antiperspirant compound. Active antiperspirant compounds contain at least one active ingredient, typically metal salts that are characterized by their ability to reduce sweating. According to the conventionally accepted mechanism, the metal salts diffuse through the sweat ducts of apocrine glands (sweat glands responsible for body odor) and hydrolyze in the sweat ducts, where they combine with proteins to form an amorphous metal hydroxide agglomerate, plugging the sweat ducts so sweat can not diffuse to the skin surface.

Some active antiperspirant compounds that may be used in the antiperspirant product include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; a and b total to about 6; x is from 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas.

Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium octachlorohydrex glycine complex. In a more preferred embodiment, the antiperspirant compound includes aluminum zirconium octachlorohydrex glycine complex at an active level of about 15 to about 25 wt. % of the total antiperspirant product.

The water phase also may include optional ingredients that serve a particular purpose. In one exemplary embodiment, the water phase includes an activator for the active antiperspirant compound. In a preferred embodiment, the water phase includes calcium chloride and in a more preferred embodiment includes calcium chloride and/or calcium carbonate in an amount of about 0.7 to about 2 wt. % of the total antiperspirant product.

The water phase further includes a water-soluble carrier in an amount that is sufficient to solubilize or disperse the water phase ingredients. The carrier includes a plurality of pre-selected polyalkylene glycols. The polyalkylene glycols are selected based on their combined capability to solubilize and to reduce or eliminate tackiness for the user of the antiperspirant emulsion product. Exemplary water-in-oil emulsion antiperspirant formulations include a plurality of polyalkylene glycols at a combined concentration ranging between about 1 wt. % and about 12 wt. % of the total antiperspirant product, and preferably ranging between about 3 wt. % and about 8 wt. %.

The present inventor discovered that in a water/oil emulsion antiperspirant formulation, tackiness imparted to a user is unexpectedly reduced or eliminated when a plurality of polyalkylene glycols are combined at a specific ratio, with each polyalkylene glycol having a molecular weight that falls in a predetermined range. Specific examples of suitable polyaklyene glycols are polyethylene glycol and polypropylene glycol. These linear polymers of ethylene and propylene oxide have the general formulas:

H[OCH₂CH₂]ₘOH (I)

H[OCHCH₃CH₂]ₙOH (II)

wherein m=5 to 20, and n=10 to 30.

According to an exemplary embodiment, n is greater than m, and preferably n is at least twice as great as m.

The ethylene oxide polymers and propylene oxide polymers are included at a ratio that ranges between about 1.5:1 and about 1:1.5. The ethylene oxide polymers and propylene oxide polymers are preferably included at a ratio that ranges between about 1.25:1 and about 1:1.25, and most preferably the ratio of the two is substantially 1:1. Within these ranges, and particularly as the ratio of ethylene oxide polymers and propylene oxide polymers approximated 1:1, tackiness substantially decreases relative to comparative formulas that included polyalkylene glycols of similar weights but at different ratios, as well as formulas that included only non-polymerized alkylene glycols.

The various embodiments of the antiperspirant emulsion products also include water and/or other volatile carrier fluids that provide a cooling effect to the user's skin when they evaporate following application thereon. As noted above, the water and/or other volatile carrier fluids evaporate from the antiperspirant product upon application of the antiperspirant product to the skin to providing the cooling sensation.

Additional carriers that may be included with the polyalkylene glycols include, but are not limited to, propylene glycol, glycerol, dipropyl glycol, ethylene glycol, butylene glycol, propylene carbonate, dimethyl isosorbide, hexylene glycol, ethanol, n-butyl alcohol, n-propyl alcohol, and isopropyl alcohol. An exemplary antiperspirant emulsion product includes non-polyalkylene glycol carriers at a concentration that is no more than 20 wt. % of the total carrier content. A preferred antiperspirant emulsion product includes such non-polyalkylene glycol carriers at no more than about 10 wt. %, and most preferably at no more than about 3 wt. %, of the total carrier content.

Further, in various embodiments, the antiperspirant emulsion products include one or more structurants that impart minimal white residue on the skin and that enable the antiperspirant emulsion products to be manufactured at lower temperatures.

In addition to the ingredients identified above, the antiperspirant product may include additives, such as those used in conventional antiperspirants. These additives include, but are not limited to, fragrances, including encapsulated fragrances, dyes, pigments, preservatives, antioxidants, emollients, moisturizers, and the like. These optional ingredients can be included in the antiperspirant product in an amount of 0 to about 20% by weight.

The antiperspirant product according to various embodiments can be prepared by combining the ingredients of the water phase using sufficient agitation to prepare a mixture and then heating the water phase. Similarly, the ingredients of the oil phase are combined and the oil phase is heated to a temperature at least sufficient to melt the structurant. The oil phase and the water phase are mixed together to form a water-in-oil emulsion. Any additive, such as fragrance, is then added to the water-in-oil emulsion. The resulting liquid antiperspirant emulsion product is poured into suitable molds and then cooled to room temperature.

The following is an exemplary embodiment of an antiperspirant product, with each of the components set forth in weight percent of the antiperspirant product. The example is provided for illustration purposes only and is not meant to limit the various embodiments of the antiperspirant product in any way.

### Example 1

| Component | wt. % |
|---|---|
| Antiperspirant active | 18-24 |
| Emollient/Residue masking agent | 2-12 |
| Oil phase structurant | 5-10 |
| Polyalkylene glycols | 2-10 |
| PEG-8 | 0.5-6 |
| PEG-17 | 0.5-6 |
| Emulsifier | 1-4 |
| AP activator | 0.7-2 |
| Synthetic wax | 0-3 |
| Fragrance | 0-2.5 |
| Water | q.s (total water phase 35-45 wt. %) |
| Oil phase carrier | q.s. (total oil phase 55-65 wt. %) |
| Total | 100 |

### Example 1a

| Component | wt. % |
|---|---|
| Aluminum Zirconium Octachlorohydrex, GLY | 17.80 |
| C12-C15 Alkyl Benzoate | 8.00 |
| Polyethylene (MW = 500) | 10.50 |
| Polyalkylene glycols | 5.00 |
| PEG-8 | 2.50 |
| PEG-17 | 2.50 |
| Cetyl PEG/PPG-10/1 dimethicone | 1.25 |
| Synthetic wax (MW = 1800) | 0.10 |
| Fragrance | 1.90 |
| Water | 43.58 |
| Cyclohexasiloxane | 11.87 |
| Total | 100 |

### Example 1b

| Component | wt. % |
|---|---|
| Aluminum Zirconium Octachlorohydrex, GLY | 17.60 |
| C12-C15 Alkyl Benzoate | 8.00 |
| Polyethylene (MW = 500) | 10.50 |
| Polyalkylene glycols | 7.32 |
| PEG-8 | 3.66 |
| PEG-17 | 3.66 |
| Cetyl PEG/PPG-10/1 dimethicone | 1.25 |
| Synthetic wax (MW = 1800) | 0.10 |
| Fragrance | 1.90 |
| Water | 41.46 |
| Cyclohexasiloxane | 11.87 |
| Total | 100 |

The antiperspirant products of the general Example 1a may be prepared by combining the water and the calcium chloride in a mixing vessel and agitating the mixture until dissolution is achieved. The polyalkylene glycols are added to this solution. Using sufficient agitation to maintain a vortex, the aluminum zirconium octachlorohydrex GLY is added to the solution and the solution heated to about 80° C. Separately, all ingredients of the oil phase are combined and the oil phase mixture is heated to a temperature in the range of about 90-100° C. until the high MW polyethylene melts. (The temperature range for melting the polyethylene may vary depending on the amount of low MW synthetic wax present in the oil phase.) The oil phase is cooled to about 80° C., the temperature is then maintained, and sufficient agitation to maintain a vortex is continued. Next, the water phase is slowly added to the oil phase by droplet at constant temperature, keeping both phases subject to agitation and between 80-85° C., to form a continuous emulsion. The speed of the agitation then is increased. For a 400 g batch of emulsion, the speed may be approximately 600 revolutions per minute (RPM). Once the water phase is fully added to the oil phase, rapid mixing and temperature are maintained for 10 minutes. The emulsion is cooled to about 80° C., the fragrance is added, and the resulting antiperspirant product is poured into a mold and allowed to cool to room temperature.

As previously mentioned, as the ratio of ethylene oxide polymers and propylene oxide polymers approximate 1:1, tackiness substantially decreases relative to comparative formulas that included polyalkylene glycols of similar weights but at different ratios, as well as formulas that included only non-polymerized alkylene glycols as carriers. For example, the antiperspirant emulsion formulation of Example 1a having a 1:1 ratio of PEG-8 and PPG-17 were compared against several comparative formulations in an in-vitro BART test in which an acrylic 0.5 in diameter probe was applied against a formulation surface at an approach speed of 0.5 mm/sec with an approach force of 1110 g for a duration of ten seconds, and then returned from the formulation surface at the same return speed. The maximum retreat force on the probe was measured. The BART test was then repeated on several comparative formulations.

The retreat force experienced by the probe for the formulation of Example 1a was less than half of the retreat force for otherwise identical formulations that included PPG-17 and PEG-8 at ratios of 1:3 and 3:1. Also, the retreat force experienced for the formulation of Example 1a was 30-40% of otherwise identical formulations that included only PEG-8, only PPG-17, only PPG-34, or only PEG-12. Identical tests were also performed with otherwise identical formulations that included propylene glycol alone or combined with varying amounts of PPG-17 and PEG-8, and the retreat force for the formulation of Example 1 was significantly less in each instance.

In vitro tests with 250 subjects were performed comparing the formulation of Example 1b with an otherwise identical formulation that included only polypropylene glycol as a carrier. The subjects applied both formulations and then reported a significant reduction in tackiness for the formulation of Example 1b both immediately upon application and throughout the day following application of the formulations. In addition, the subjects reported significant improvements for other subjective measures including glide, wetness of application, texture of application, amount of residue, whiteness of any residue, caking of residue, evenness of coverage, slipperiness after application, and slipperiness throughout the day following application.

Accordingly, antiperspirant products with antiperspirant efficacy that provide numerous advantages to a user of the products, and processes for making such antiperspirant products, have been provided. The various embodiments of the antiperspirant emulsion products also include water and volatile carrier fluids that provide a cooling effect to the skin when they evaporate upon application to the skin. Further, in various embodiments, the antiperspirant emulsion products include a unique combination of structurants that result in less white residue on the skin and that cause the antiperspirant emulsion products to be manufacturable at lower temperatures. Most importantly, the products surprisingly impart highly reduced tackiness to the user.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A water in oil emulsion composition, comprising:
an antiperspirant active;
an emulsifier; and
a combination of polyalkylene glycols making up between about 1 and about 12 wt. % of the total composition, the combination comprising:
H[OCH₂CH₂]ₘOH (I), and
H[OCHCH₃CH₂]ₙOH (II)
in a weight ratio I:II ranging between about 1.25:1 and about 1:1.25.

2. The water in oil emulsion composition according to claim 1, wherein among the polyalkylene glycols, m=5 to 20, and n=10 to 30.

3. The water in oil emulsion composition according to claim 2, wherein among the polyalkylene glycols, n is greater than m.

4. The water in oil emulsion composition according to claim 3, wherein among the polyalkylene glycols, n is at least twice as great as m.

5. (canceled)

6. The water in oil emulsion composition according to claim 1, wherein the ratio I:II is substantially 1:1.

7. The water in oil emulsion composition according to claim 1, wherein the emulsifier comprises a polyoxyalkylene modified dimethylpolysiloxane.

8. The water in oil emulsion composition according to claim 7, wherein the emulsifier comprises cetyl PEG/PPG 10/1 dimethicone.

9. The water in oil emulsion composition according to claim 1, further comprising a volatile carrier fluid that provides a cooling sensation to a user's skin upon application of the antiperspirant product to the skin.

10. The water in oil emulsion composition according to claim 1, wherein the combination of polyalkylene glycols make up between about 3 wt. % and about 8 wt. % of the total composition.

11. A water in oil emulsion composition, comprising:
an antiperspirant active;
an emulsifier; and
a combination of polyalkylene glycols making up between about 1 and about 12 wt. % of the total composition, the combination comprising:
H[OCH₂CH₂]ₘOH (I), and
H[OCHCH₃CH₂]ₙOH (II)
in a weight ratio I:II that is substantially 1:1, wherein m=5 to 20, and n=10 to 30 and n is at least twice as great as m.

12. The water in oil emulsion composition according to claim 11, wherein the emulsifier comprises cetyl PEG/PPG 10/1 dimethicone.

13. The water in oil emulsion composition according to claim 11, further comprising a volatile carrier fluid that provides a cooling sensation to a user's skin upon application of the antiperspirant product to the skin.

14. The water in oil emulsion composition according to claim 11, wherein the combination of polyalkylene glycols make up between about 3 wt. % and about 8 wt. % of the total composition.
